(19) United States
(12) Patent Application Publication
(10) Pub. No.: US 2024/0269470 A1
(43) Pub. Date: Aug. 15, 2024

(54) NEUROSTIMULATION OF MIXED NERVES

(71) Applicant: Saluda Medical Pty Ltd, Macquarie Park (AU)

(72) Inventors: John Louis Parker, Artarmon (AU); Gerritt Eduard Gmel, Artarmon (AU)

(21) Appl. No.: 18/443,609

(22) Filed: Feb. 16, 2024

Related U.S. Application Data
(63) Continuation of application No. 17/050,788, filed on Oct. 26, 2020, now Pat. No. 11,944,820.

(30) Foreign Application Priority Data
Apr. 27, 2018 (AU) 2018901410

(51) Int. Cl. A61N 1/36; A61N 1/05; G16H 20/40

(52) U.S. Cl. CPC A61N 1/36139; A61N 1/0553; A61N 1/0556; A61N 1/36003; A61N 1/36007; A61N 1/3601; A61N 1/36064; A61N 1/36071; A61N 1/36096; A61N 1/36114; A61N 1/36157; A61N 1/36171; A61N 1/36175; G16H 20/40

(57) ABSTRACT

Neurostimulation of a mixed nerve comprising a plurality of nerve fibre types. An implantable electrode array comprising a plurality of electrodes is positioned proximal to the mixed nerve. An electrical stimulus is delivered from at least one nominal stimulus electrode of the implantable electrode array, in accordance with a set of stimulus parameters. A recording of the electrophysiological response evoked by the electrical stimulus is obtained from at least one nominal recording electrode of the implantable electrode array. The recording is analysed by assessing one or more selected characteristics of the recording, and from the observed selected characteristics a level of recruitment of one or more fibre types recruited by the electrical stimulus is identified. The stimulus parameters are refined in a manner to effect selective recruitment of one or more fibre types relative to other fibre types of the mixed nerve.

### CROSS-REFERENCE TO RELATED APPLICATIONS

This application is a continuation of U.S. patent application Ser. No. 17/050,788, filed on Oct. 26, 2020, which is a national stage of International Application No. PCT/AU2019/050384, filed on Apr. 29, 2019, which claims the benefit of Australian Provisional Patent Application No. 2018901410, filed Apr. 27, 2018, which applications are incorporated herein by reference in their entirety.

### TECHNICAL FIELD

The present invention relates to neuromodulation delivered to mixed nerve fibres comprising multiple fibre types, and in particular to a method and device for assessing recruitment of a desired subset of the fibre types from electrophysiological response measurements.

### BACKGROUND OF THE INVENTION

There are a range of situations in which it is desirable to apply electrical stimuli to a nerve in order to give rise to a compound action potential (CAP). A neuromodulation system applies an electrical pulse to tissue in order to generate a therapeutic effect. Such a system typically comprises an implanted electrical pulse generator, and a power source such as a battery that may be rechargeable by transcutaneous inductive transfer. An electrode array is connected to the pulse generator, and is positioned adjacent the target neural pathway(s). An electrical pulse applied to the neural pathway by an electrode causes the depolarisation of neurons, and generation of propagating action potentials.

Almost all applications of neuromodulation are applied to nerves containing more than one type of fibre (referred to herein as a “mixed nerve”). It is often the case that a large proportion of the fibres of a mixed nerve, when stimulated, do not produce an effect that is directly and immediately perceivable by the subject or an outside observer (such as a surgeon or clinician). For example, stimulation of fibres of the autonomic nervous system is often not perceptible by the subject.

A control problem, facing neuromodulation systems of all types, is achieving neural recruitment at a sufficient level required for therapeutic effect, but at minimal expenditure of energy. The power consumption of the stimulation paradigm employed has a direct effect on battery requirements which in turn affects the device's physical size and lifetime. For rechargeable systems, increased power consumption results in more frequent charging and, given that batteries only permit a limited number of charging cycles, ultimately this reduces the implanted lifetime of the device.

One example of neuromodulation of a mixed nerve is sacral nerve stimulation (SNS), in which stimulation frequencies are typically low (<20 Hz) and the charge can be quite high (for example, each stimulus may comprise a current of up to 7 mA or more, at pulse widths of 210 us). SNS has been shown to be therapeutically effective for faecal incontinence (FI), Urinary Retention (UR), Urinary Urge Incontinence (UUI, also referred to as overactive bladder (OAB)), intractable constipation, and chronic pelvic pain, with further indications likely.

The mechanisms of SNS are still poorly understood and various theories have been proposed. For existing sacral nerve neuromodulators, following implantation the process of adjusting the stimulus amplitude and frequency is a trial and error procedure, with muscle contractions in the form of the motor response of the pelvic floor, anal sphincter, and/or the toe being used as a proxy for therapeutic efficacy. In this testing method the stimulus amplitude is turned up until a muscle response is visually sighted intraoperatively. The amplitude is then reduced below sensation threshold and set to that level for ongoing operation, but how much reduction is adequate to avoid undesirable motor responses or paraesthesia while still maintaining appropriate therapeutic effect is poorly known. This method relies on a theory that SNS acts to re-establish sphincter control through stimulation of the efferent motor fibres, or via an afferent reflex arc. One theory is that SNS induces a reflex inhibitory effect on the detrusor muscle of the urinary bladder through afferent and efferent fibres in the sacral nerves. Another proposed mechanism, especially for genitourinary disorders is via inhibition of bladder contractions via afferent or central mechanisms.

To have a SNS device operating continuously at amplitude levels just below the muscle or paraesthesia recruitment threshold involves a considerable power drain on the implant battery.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood that the element may be any one of the listed options, or may be any combination of two or more of the listed options.

### SUMMARY OF THE INVENTION

According to a first aspect, the present invention provides a method of neurostimulation of a mixed nerve comprising a plurality of nerve fibre types, the method comprising:
positioning an implantable electrode array proximal to a mixed nerve comprising a plurality of nerve fibre types, the implantable electrode array comprising a plurality of electrodes;
delivering an electrical stimulus from at least one nominal stimulus electrode of the implantable electrode array, the electrical stimulus being delivered in accordance with a set of stimulus parameters;
obtaining from at least one nominal recording electrode of the implantable electrode array a recording of the electrophysiological response evoked by the electrical stimulus;
analysing the recording of the electrophysiological response by assessing one or more selected characteristics of the recording, and identifying from the observed selected characteristics a level of recruitment of one or more fibre types recruited by the electrical stimulus; and
refining the stimulus parameters in a manner to effect selective recruitment of one or more fibre types relative to other fibre types of the mixed nerve.

According to a second aspect, the present invention provides a non-transitory computer readable medium for neurostimulation of a mixed nerve comprising a plurality of nerve fibre types, comprising instructions which, when executed by one or more processors, causes performance of the following:
delivering an electrical stimulus from at least one nominal stimulus electrode of an implantable electrode array proximal to a mixed nerve comprising a plurality of nerve fibre types, the implantable electrode array comprising a plurality of electrodes, the electrical stimulus being delivered in accordance with a set of stimulus parameters;
obtaining from at least one nominal recording electrode of the implantable electrode array a recording of the electrophysiological response evoked by the electrical stimulus;
analysing the recording of the electrophysiological response by assessing one or more selected characteristics of the recording, and identifying from the observed selected characteristics a level of recruitment of at least a first fibre type recruited by the electrical stimulus; and
refining the stimulus parameters in a manner to effect selective recruitment of one or more fibre types relative to other fibre types of the mixed nerve.

According to a third aspect the present invention provides a neurostimulation device comprising:
an implantable electrode array configured to be implanted proximal to a mixed nerve comprising a plurality of nerve fibre types, the implantable electrode array comprising a plurality of electrodes; and
a control unit configured to deliver an electrical stimulus from at least one nominal stimulus electrode of the implantable electrode array, the electrical stimulus being delivered in accordance with a set of stimulus parameters; the control unit further configured to obtain from at least one nominal recording electrode of the implantable electrode array a recording of the electrophysiological response evoked by the electrical stimulus; the control unit further configured to analyse the recording of the electrophysiological response by assessing one or more selected characteristics of the recording, and identify from the observed selected characteristics a level of recruitment of one or more fibre types recruited by the electrical stimulus, and the control unit further configured to refine the stimulus parameters in a manner to effect selective recruitment of one or more fibre types relative to other fibre types of the mixed nerve.

Importantly, the present invention thus utilises one or more recordings of electrically evoked electrophysiological response(s) obtained from a nerve proximal to a stimulation site as a means to selectively deliver neurostimulation to a selected fibre type selected from a plurality of fibre types existing in the nerve.

Throughout this specification, the term “electrophysiological response” is to be understood as including one or more neural responses (CAPs), myoelectric responses (such as motor unit action potentials and compound muscle action potentials (CMAPs)), and/or interneuron activity (the firing of neurons that do not possess long axonal projections such as sensory fibres). A neural response evoked by an applied stimulus is also referred to herein as an evoked compound action potential (ECAP). In addition to a neural response, a recording of an electrophysiological response to a stimulus can also include myoelectric activity, also referred to in some instances herein as a late response.

Some embodiments of the present invention may additionally or alternatively be advantageous over past approaches which target for example activation of a particular muscle, or muscle group, such as the anal sphincter, such as by intraoperative observation of muscle activation for the purpose of assessing the proximity of the stimulus electrode to the mixed nerve. By seeking muscle activation alone, or a proxy thereof, as the goal of neurostimulation, such past approaches are blind as to which fibre types are recruited by the stimuli, so long as muscle activation is observed. In contrast to such past approaches, the present invention's approach of targeting one or more specific fibre types on the basis of observed electrophysiological response measurements avoids the obfuscating effect of the propagation of the stimulated neural response away from the stimulus site to a muscle, as such propagation typically passes a range of neural branches, synapses, terminations, and further involves muscle fibre activation by motor neurons, all of which results in muscle observations being at least partly blinded to the nature of the neural recruitment occurring at the stimulus site. Moreover the present invention's approach of targeting specific fibre types on the basis of observed electrophysiological response measurements further opens the possibility of, in some embodiments, delivering therapy on the basis of recruitment of fibres unrelated to muscle activation and thus wholly undetectable by muscle observations. For example neuromodulation of fibres of the autonomic nervous system may not be perceptible by the patient nor by external clinical observation. Such embodiments of the present invention thus recognise that in order to optimise neuromodulation of a mixed nerve, an objective measure of the recruited fibres needs to be used.

In some embodiments of the invention, the stimulus parameters which are refined to effect selective recruitment of one or more fibre types may comprise any one or more of: intraoperative electrode placement; intraoperative electrode array type selection, including lead, paddle or cuff array selection; stimulus frequency; stimulus amplitude; stimulus waveform; stimulus pulse width; stimulus electrode(s) selection, including interposition of a stimulus site between electrodes by way of current steering; stimulus shape (biphasic, triphasic, etc); stimulus polarity (monopolar, bipolar, tripolar, etc), stimulus electrode size, stimulus electrode shape, and the like.

In some embodiments of the invention, the stimulus parameters are refined in a manner to effect selective recruitment of one or more desired fibre types while further effecting selective non-recruitment or diminished recruitment of at least one non-selected fibre type.

The one or more selected characteristics of the recording from which the level of recruitment of at least the first fibre type recruited by the electrical stimulus is identified may comprise any one or more of: one or more ECAP inflexion points; one or more ECAP peak positions; one or more ECAP peak amplitudes; ECAP propagation velocity; propagation or non-propagation of an electrophysiological response as observed at a recording site; ECAP duration; refractory period; strength-duration curve characteristics including chronaxie or rheobase; growth curve characteristics including threshold and slope; number of ECAP peaks with increasing stimulus current; presence, amplitude and/or latency of a late response, amplitude and shape of the electrophysiological response with varying stimulus frequency.

For example, in embodiments where the selected characteristic is conduction velocity, the fibre type recruited may be determined from a priori knowledge of the linear relationship between the diameter of a myelinated fibre and the conduction velocity.

In embodiments where the selected characteristic is conduction velocity, the conduction velocity may be measured by determining a propagation time from the stimulus site to a single measurement electrode a known distance from the stimulus site. More preferably, the conduction velocity may be measured by observing a neural response at a first measurement electrode and at a second measurement electrode, and determining a propagation time between the first and second measurement electrodes. Determining conduction velocity from two or more measurement electrodes allows inspection of particular elements of the ECAP waveform, such as a peak arrival time or a zero crossing arrival time of the ECAP, improving accuracy of the conduction velocity determination and in turn improving accuracy of identification of the recruited fibre type.

In some embodiments of the present invention, with prior knowledge of the morphology of neural responses under different fibre distributions, models can be used to solve the reverse problem of retrieving the fibre distribution from one or more obtained recordings of electrophysiological responses evoked by electrically stimulating a mixed nerve. For example the teachings of the present Applicant's International Patent Publication No. WO2016161484 (PCT/AU2016/050263) relating to fibre distribution modelling may be applied to this purpose.

The selected characteristic may be determined by assessment of recordings obtained from two or more spaced apart measurement electrodes, the recordings being of a single electrophysiological response event. Obtaining spatially distinct recordings of the same electrophysiological response for example allows determination of whether, relative to the vicinity of the recording electrodes, the selected characteristics of the recordings are a propagating neural response upon the mixed nerve or a non-propagating response such as myoelectric activity in the far field of the electrodes, thereby assisting fibre type determination. While it is to be noted that recordings obtained in the close vicinity of an activated muscle will observe a propagating CMAP, present embodiments of the invention will typically utilise an electrode array implanted distally from affected muscles so that myoelectric activity will in such embodiments be observed as a non-propagating component of the recordings of the electrophysiological response.

In embodiments where the selected characteristic is a non-propagating characteristic of the recording, an existence of such a non-propagating component in the recording, such as a component arising 4-10 ms after stimulus, may be taken to arise from the myoelectric activity produced by the stimulus by, for example, stimulating motor neurons. The muscle activation may thus be deduced to arise from activation of Aα efferent fibres, which having a high conduction velocity may not be directly observable in the recordings, as Aα responses may have concluded at the recording electrode before the stimulus is complete or before stimulus artefact has settled sufficiently to permit direct observation of Aα fibre responses. However, selective Aα recruitment may be observed by reference to the non-propagating component arising 4-10 ms after stimulus in the recording (for SNS, at least). The amplitude of such a non-propagating component may thus be taken as a measure of the number of Aα fibres recruited, permitting selective recruitment of Aα fibres.

Further embodiments of the invention may provide for differentiation of a mode of activation of one or more recruited fibre types. For example, without intending to be limited by theory, it is noted that activation of Aα fibres may be the result of either direct activation by the electrical stimulus, or the result of indirect activation via a reflex arc such as the H-reflex elicited when stimulating Ia proprioceptive fibres. The mode of activation can be difficult to ascertain given that Ia fibres have substantially the same conduction velocity as Aα fibres and thus cannot be distinguished by this measure. However, it is further noted that with increasing stimulation frequency Aα fibre activation by the H-reflex will decline at relatively low frequencies such as at around 30 Hz stimulation rate, whereas direct Aα fibre activation does not decline until a higher stimulation frequency is reached. Accordingly, some embodiments of the invention may provide for identifying a mode of activation of one or more fibre types, by applying varied stimulation rates, identifying a threshold frequency above which activation declines, and determining from the threshold frequency a mode of activation.

In some embodiments the selected characteristic may comprise a propagating response arising in the recording less than 1 ms after the stimulus and/or having a conduction velocity in the range 80-120 m/s, taken to indicate activation of Aα fibres.

In some embodiments the selected characteristic may comprise a propagating response arising in the recording less than 6 ms after the stimulus and/or having a conduction velocity in the range 3-15 m/s, taken to indicate activation of B fibres.

In some embodiments the selected characteristic may comprise a propagating response arising in the recording less than 6 ms after the stimulus and/or having a conduction velocity in the range 0.5-2 m/s, and/or having a duration of over 10 ms, taken to indicate activation of C fibres.

In some embodiments the selected characteristic may comprise a propagating response arising in the recording within 3 ms after the stimulus and/or having a conduction velocity in the range 30-80 m/s, taken to indicate activation of Aβ fibres.

In some embodiments the selected characteristic may correspond to any fibre type of interest based on known characteristics of such a fibre type, as defined by any suitable fibre classification system.

In some embodiments, more than one selected characteristic of the recording of the electrophysiological response may be assessed in order to determine a level of recruitment of each of one or more fibre types recruited by the electrical stimulus. For example, a level of recruitment of motor fibres may be determined by reference to both an amplitude of a late response in the recording, and also by reference to whether the late response is non-propagating between multiple recording electrodes.

In some embodiments, two or more fibre types may be targeted for example for the purpose of more effectively treating a single condition, and/or to simultaneously treat two or more co-existing or comorbid conditions, for example where each fibre type is therapeutic in relation to a respective condition. For example, one of a sympathetic nerve fibre type and a parasympathetic nerve fibre type may be targeted to excite activity of a selected organ or body system at a first time, and the other of the sympathetic nerve fibre type and the parasympathetic nerve fibre type may be targeted to inhibit activity of the selected organ or body system at a second time.

A plurality of selected characteristics, or in some embodiments all of the above described selected characteristics, may be monitored in the recording(s) of the electrophysiological response(s). In some embodiments a machine learning classifier may be applied in order to classify observed electrophysiological responses by fibre type(s) present.

In some embodiments of the invention, the electrode array comprises a single implantable lead. In some embodiments of the invention, the electrode array comprises a plurality of electrode leads connected to a single implantable pulse generator (IPG).

In some embodiments of the invention, the at least one nominal recording electrode, and the at least one nominal stimulus electrode, are positioned adjacent to a single branch of the mixed nerve, being a segment of the mixed nerve in which no neural branching or neural merging occurs upon the nerve between the nominal recording electrode(s) and the nominal stimulus electrode(s).

In some embodiments of the invention, the nominal recording electrode(s) and the nominal stimulus electrode(s) are positioned less than 60 mm apart. In some embodiments of the invention, the nominal recording electrode(s) and the nominal stimulus electrode(s) are positioned less than 30 mm apart. In some embodiments of the invention, the nominal recording electrode(s) and the nominal stimulus electrode(s) are positioned less than 20 mm apart. Positioning the recording electrodes close to the stimulus site is advantageous in improving understanding of the recruitment of the one or more fibre types resulting from the stimulus, before the nerve response passes to another vertebral segment or passes a synapse or ganglion, for example.

A mixed nerve is defined herein as including a nerve comprising at least two fibre types. Fibre types are defined herein as fibres having distinct diameter, conduction velocity, myelination, efference or afference, nervous sub-system (e.g. sympathetic, parasympathetic) or other such distinguishable characteristic. For example the fibre types may be distinct based on comprising two or more of Aα, Aβ, Aγ, Aδ, B, and C fibre types, or Ia, Ib, II, III and IV fibre types. The mixed nerve may comprise part of the central nervous system, or a part of the peripheral nervous system. The mixed nerve may comprise part of the somatic nervous system, or part of the autonomic nervous system, or both. The mixed nerve may be wholly afferent or wholly efferent, or may comprise both afferent and efferent fibres. The mixed nerve may comprise fibres which carry sensory information, motor information, or both. The mixed nerve may comprise fibres which are part of one or more of the sympathetic nervous system, the parasympathetic nervous system and the enteric nervous system.

It is further to be understood herein that the mixed nerve may comprise more than one nerve, such as a plurality of adjacent nerves comprising a plurality of nerve fibre types. Thus, some embodiments of the invention may comprise determining which nerve within a plurality of adjacent nerves contains the fibres that a given neuromodulation applications aims to stimulate. The plurality of adjacent nerves may comprise a nerve plexus, such as the sacral plexus or the brachial plexus. Electrode placement and stimulus parameters can in such embodiments then be adapted to optimally recruit the desired fibre types while minimising recruitment of undesired fibre types. For example, although generally consistent, the human anatomy can differ from person to person and some differences in innervation is common. While past approaches may operate for example on an anatomical assumption that a given site, such as the S3 foramen, is a most appropriate site for neuromodulation, the present invention instead provides for an objective determination of which stimulus site is most effectively recruiting one or more fibres types of interest, so that a location of stimulation may be refined accordingly. Such embodiments of the present invention therefore allow personalised therapies to be developed that take into consideration the subject's anatomy.

The mixed nerve may comprise the vagus nerve. In such embodiments the first fibre type preferentially recruited may comprise parasympathetic fibres, such as B fibres, to provide a therapy for a brain related condition such as refractory epilepsy or depression. Additionally or alternatively vagus nerve stimulation may be configured to preferentially recruit B fibres in order to serve a therapeutic effect in the periphery or viscera such as an anti-inflammatory effect, for example to influence the spleen to alter the immune response, such as to treat Crohn's disease or rheumatoid arthritis, or a condition of the liver.

Additionally or alternatively, in embodiments targeting the vagus nerve, the stimulation may be configured to reduce or avoid recruitment of any one or more of: Aβ fibres to avoid tingling throat side effects; Aα fibres to avoid hoarseness and voice alteration (difficulty speaking, dysphonia, etc) side effects; C fibres to avoid pain side effects, and Aδ fibres to avoid pain side effects. Preferred embodiments selectively recruit each of these plurality of fibre types of the vagus nerve only to a degree which is therapeutic while avoiding or minimising side effects.

In some embodiments one or more fibre types of the vagus nerve may be targeted in order to treat one or more of: obesity, epilepsy, paced stomach (gastric reflux), pancreatitis, diabetes, inflammatory bowel disease, rheumatoid arthritis, Crohn's disease, fibromyalgia, other inflammatory disease, depression, sepsis, or pain (fibromyalgia, migraines).

In some embodiments one or more proprioceptive or motor fibre types of the dorsal roots and/or dorsal columns may be targeted in order to treat one or more of spasticity, Parkinson's disease, or other motor control disorders.

In some embodiments one or more fibre types may be targeted in order to treat a disorder of the autonomic nervous system, such as dysregulation of the bladder, the digestive system, the heart or the blood vessels.

In some embodiments one or more fibre types of the phrenic nerve may be targeted in order to treat a breathing disorder to induce paced diaphragm contractions.

In some embodiments one or more fibre types of the tibial nerve may be targeted in order to treat bladder control disorders.

In some embodiments one or more motor fibre types may be targeted through functional electrical stimulation in order to treat a motor control dysfunction or to effect rehabilitation.

In some embodiments the relative activation of the postsynaptic dorsal column pathway and the primary sensory afferents may be optimised in order to treat neuropathic pain.

The mixed nerve may in some embodiments comprise the sacral nerve. In such embodiments the first fibre type preferentially recruited may comprise parasympathetic fibres, or other fibre types. Sacral nerve stimulation may be provided to selected fibre types to provide a therapy for one or more of faecal incontinence (FI), Urinary Retention (UR), Urinary Urge Incontinence (UUI, also referred to as overactive bladder (OAB)), intractable constipation, and chronic pelvic pain.

The mixed nerve may in some embodiments comprise a preganglionic mixed nerve such as the vagus nerve, a ventral root, or the sacral nerve. Such embodiments are advantageous in permitting stimulation to occur at a site which is relatively easy to access, and at which relatively low stimulation intensity is required, while selectively recruiting only the fibres of interest. This is in contrast to past approaches targeting post-ganglionic C nerves which are difficult to access and which require high stimulus intensity.

In some embodiments the mixed nerve may comprise a root of a spinal nerve such as a ventral root. The ventral root may comprise motor fibres and parasympathetic fibres. The stimulation may in some embodiments be configured in order to preferentially recruit the motor fibres of the ventral root in order to directly activate motor neuron fibre(s) of interest.

The stimulation may in some embodiments be configured in order to preferentially recruit parasympathetic and/or sympathetic fibres of a preganglionic mixed nerve.

Parasympathetic stimulation may be targeted to any one or more of the heart, larynx, trachea, bronchi, oesophagus, stomach, liver, pancreas, small intestine, spleen, large intestine or kidney, all originating from the 10th cranial nerve, also referred to as the vagus nerve. Parasympathetic stimulation may be targeted to any one or more of the large intestine, bladder, and genitalia all originating from the sacral segments of the spinal cord. Sympathetic nerve stimulation may be targeted to the heart and/or larynx by stimulating the sympathetic fibres in one or more of the ventral roots of thoracic segments T1-T4. Sympathetic nerve stimulation may be targeted to the stomach, liver, pancreas, adrenal gland, spleen, and/or small intestine by stimulating the sympathetic fibres in one or more of the ventral roots of thoracic segments T5-T12. Sympathetic nerve stimulation may be targeted to the kidney, bladder, genitalia, and/or lower intestine by stimulating the sympathetic fibres in one or more of the ventral roots of thoracic segments T11-T12 and the lumbar segments L1-L3. For example, some embodiments may provide sympathetic and parasympathetic stimulation of the liver by stimulating the sympathetic fibres in the thoracic ventral roots T5-T12 and the parasympathetic fibres originating in the vagus nerve.

In some embodiments of the invention refining the stimulus parameters comprises a clinical fitting process of stimulus parameters by clinician trial and error or the like. Refining the stimulus parameters may in some embodiments comprise intraoperative repositioning of the electrodes.

Refining the stimulus parameters in other embodiments may comprise an automated feedback process administered by a processor of the implanted device.

Some embodiments may spatially target the selected fibre type, by applying a supramaximal stimulus from a first electrode to recruit all fibres of the nerve, and observing the recruited responses at selected circumferential positions by using a selected electrode segment for recording at the selected circumferential position, analysing the recorded response to determine one or more fibre types which are adjacent to that position, and subsequently applying stimuli from the selected electrode segment at times when it is desired to recruit the one or more fibre types so identified. Other embodiments may spatially target the selected fibre type by using a selected electrode segment at a selected circumferential position to apply stimuli which are only just above a stimulus threshold to recruit fibres proximal to that segment, observing recruited responses at a second electrode, and analysing the recordings to determine the type of fibres being recruited by the stimuli from the selected electrode segment. Such embodiments may survey multiple electrode segments in this manner to determine the fibre type(s) adjacent to all such electrode segments. Other embodiments may apply equivalent spatial targeting by using electrodes which spatially differ other than in a circumferential manner, such as in a grid pattern or any other form of spatial electrode variations which permit distinct electrodes to recruit distinct subgroups of fibres.

In some embodiments the device is fully implantable and comprises an implantable pulse generator configured to deliver the stimuli via the stimulus electrodes, and to capture and analyse the recordings of the evoked electrophysiological responses to effect fibre type targeting. In alternative embodiments, the electrode array alone may be temporarily implantable, with an external control device effecting the fibre type targeting.

It is to be appreciated that a time of occurrence of electrophysiological responses in a recording is generally referred to herein by reference to an amount of time after the stimulus. However, this amount of time depends on both the conduction velocity of the fibre(s) being observed, and the distance of the respective recording electrode from the stimulus site. It is to be understood that time periods presented herein may be particularly applicable to a single implanted lead having recording electrodes spaced about 6 mm, 12 mm and 18 mm from the stimulus electrode. However, alternative electrode array geometries and configurations may provide electrodes at other distances from the stimulus site, and a simple calculation based on conduction velocity allows an alternative expected time of arrival of responses of each given fibre type to be determined, and such alternatives are within the scope of the present invention. Similarly, where a late response arising from far field activation of a muscle is a selected characteristic of interest, a time of occurrence of the late response will depend on a distance from the stimulus site to the muscle, and variations in a time of occurrence of such a late response are thus also simply determined from the stimulus site and associated anatomy, and such variations in the time of occurrence of the late response are within the scope of the present invention.

In some embodiments of the device of the third aspect of the invention the control unit is further configured to refine the stimulus parameters in a manner to effect selective recruitment of one or more fibre types relative to other fibre types of the mixed nerve. In some embodiments of the device of the third aspect of the invention the device is an implantable device, while in other embodiments the device may be an external device for trial or intraoperative use.

### BRIEF DESCRIPTION OF THE DRAWINGS

An example of the invention will now be described with reference to the accompanying drawings, in which:

FIG. 1 schematically illustrates an implanted sacral nerve stimulator;

FIG. 2 is a block diagram of the implanted neurostimulator;

FIG. 3 is a schematic illustrating interaction of the implanted stimulator with a nerve;

FIG. 4 illustrates the typical form of an electrically evoked compound action potential (ECAP) of a healthy subject;

FIG. 5 is a schematic representation of the functional separation of fibres in the ventral rootlets of a human S2 nerve in cross-section.

FIG. 6, which is a three-dimensional (3D) reconstruction of median nerve fascicles.

FIG. 7 shows the neural responses recorded from the cervical vagus nerve of a pig from a recording cuff electrode, at varying stimulus amplitude

FIGS. 8a and 8b show electrophysiological responses obtained from the S3 sacral nerves of 2 human patients undergoing SNS therapy, from differing recording electrodes along the lead

FIG. 9 illustrates sympathetic and parasympathetic nerve pathways which form part of mixed nerve neural pathways upon which the approach of the present invention may be applied.

FIGS. 10a and 10b illustrate changes in amplitude of the A3 response, and changes in amplitude of the late response, respectively, over time;

FIG. 11a illustrates measurements of the growth curve of the neural A3 response of the same human SNS patient as FIG. 10. FIG. 11b illustrates measurements of the late response growth curve. FIG. 11c illustrates recordings from a human patient, and Figure lid shows the growth curve for the late response; FIG. 11e shows the growth curve of a B fibre response in one patient with an arrow marking the current threshold for stimulus perception.

FIG. 12 illustrates changes in the amplitude of myoelectric responses observed in a human SNS patient over time;

FIG. 13 illustrates an embodiment of the invention employing multiple electrode leads for fibre type targeting;

FIG. 14 illustrates an embodiment of the invention employing a single electrode lead for fibre type targeting;

FIG. 15 illustrates another embodiment of the invention employing multiple electrode leads for fibre type targeting;

FIG. 16 illustrates an embodiment of the invention employing cuff electrodes for fibre type targeting;

FIGS. 17 and 18 illustrate fibre type targeting flowcharts.

### DESCRIPTION OF THE PREFERRED EMBODIMENTS

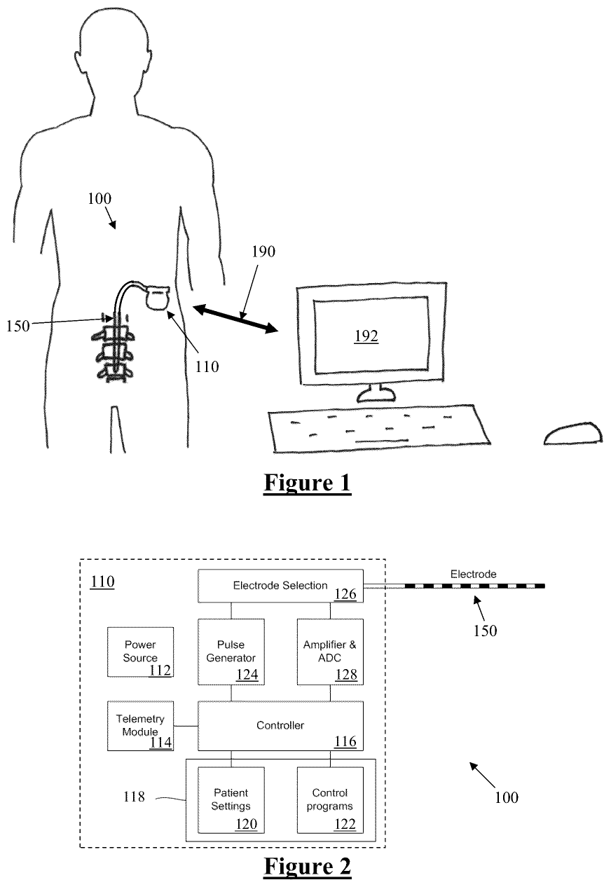

FIG. 1 schematically illustrates an implanted sacral nerve stimulator 100. Stimulator 100 comprises an electronics module 110 implanted at a suitable location in the patient's lower abdominal area or posterior superior gluteal region, and an electrode assembly 150 implanted within the sacrum and connected to the module 110 by a suitable lead. Numerous aspects of operation of implanted neural device 100 are reconfigurable by an external control device 192. Moreover, implanted neural device 100 serves a data gathering role, with gathered data being communicated to external device 192.

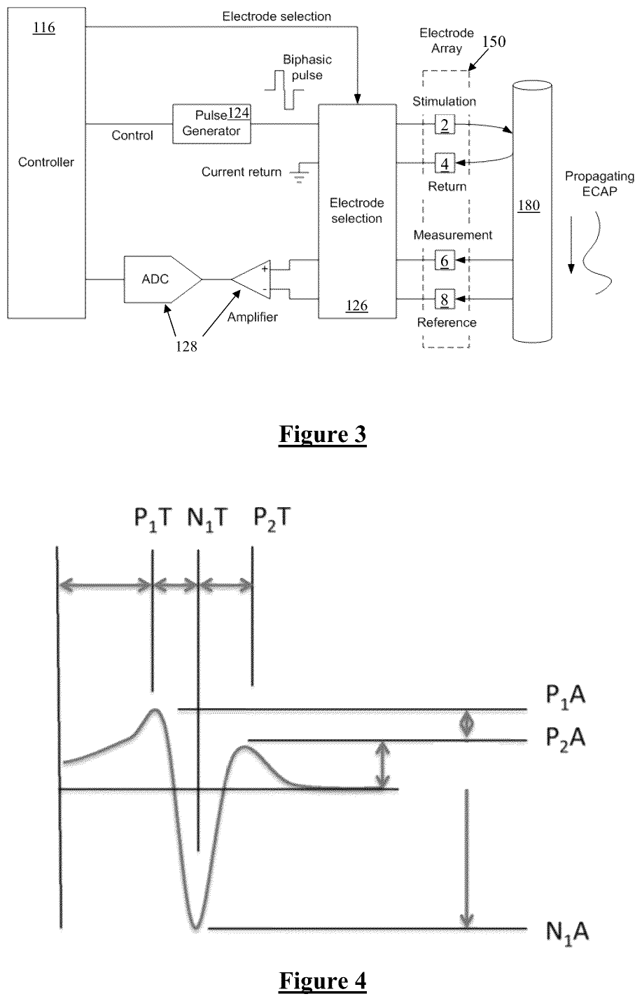

FIG. 2 is a block diagram of the implanted neurostimulator 100. Module 110 contains a battery 112 and a telemetry module 114. In embodiments of the present invention, any suitable type of transcutaneous communication 190, such as infrared (IR), electromagnetic, capacitive and inductive transfer, may be used by telemetry module 114 to transfer power and/or data between an external device 192 and the electronics module 110.

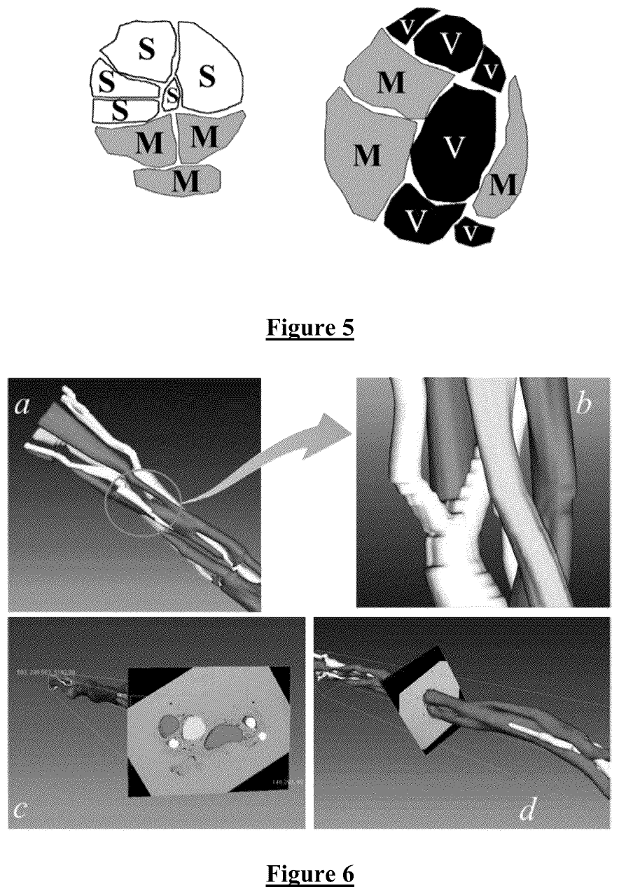

Module controller 116 has an associated memory 118 storing patient settings 120, control programs 122 and the like. Controller 116 controls a pulse generator 124 to generate stimuli in the form of current pulses in accordance with the patient settings 120 and control programs 122. Electrode selection module 126 switches the generated pulses to the appropriate electrode(s) of electrode array 150, for delivery of the current pulse to the tissue surrounding the selected electrode(s). Other electrode arrays may also be provided and may be similarly addressed by electrode selection module 126, for example as in the case of FIGS. 5 and 6, discussed further below. Thus, one or more electrodes of array 150 may be selected to serve as nominal stimulus electrodes at a given time, while one or more electrodes of the array 150 may be selected to serve as nominal sense electrodes at a given time, even though the electrodes may be physically the same and may serve a different role at other times. Measurement circuitry 128 is configured to capture measurements of neural responses sensed at sense electrode(s) of the electrode array as selected by electrode selection module 126. Such measurements will often comprise differential measurements between two sense electrodes upon array 150. However the measurements may additionally or alternatively be obtained from a single sense electrode of array 150 electrically referenced to a reference electrode upon a case of the module 110, or referenced to a system ground of the controller 116, for example. The sense electrodes are also referred to herein as recording electrodes.

FIG. 3 is a schematic illustrating interaction of the implanted stimulator 100 with a nerve 180, in this case the sacral nerve however alternative embodiments may be positioned adjacent any desired neural tissue including a peripheral nerve, visceral nerve, spinal nerve or a brain structure. Electrode selection module 126 selects a stimulation electrode 2 of electrode array 150 to deliver an electrical current pulse to surrounding tissue including nerve 180, and also selects a return electrode 4 of the array 150 for stimulus current recovery to maintain a zero net charge transfer.

Delivery of an appropriate stimulus to the nerve 180 evokes a neural response comprising a compound action potential which will propagate along the nerve 180 as illustrated, for therapeutic purposes which in the case of a sacral nerve stimulator might be to stimulate motor function of desired muscle fibres of the detrusor. To this end the stimulus electrodes are used to deliver stimuli at <20 Hz.

The device 100 is further configured to sense the existence and intensity of compound action potentials (CAPs) propagating along nerve 180, whether such CAPs are evoked by the stimulus from electrodes 2 and 4, or otherwise evoked. To this end, any electrodes of the array 150 may be selected by the electrode selection module 126 to serve as measurement electrode 6 and measurement reference electrode 8. Signals sensed by the measurement electrodes 6 and 8 are passed to measurement circuitry 128, which for example may operate in accordance with the teachings of International Patent Application Publication No. WO2012155183 by the present applicant, the content of which is incorporated herein by reference.

FIG. 4 illustrates the typical form of an electrically evoked compound action potential (ECAP) when comprised of the contributions from action potentials of recruited fibres with similar properties. The shape and duration of the compound action potential shown in FIG. 4 is predictable because it is a result of the ion currents produced by the ensemble of axons generating action potentials in response to stimulation. The action potentials generated among a large number of fibres sum to form a compound action potential (CAP). The CAP is the sum of responses from a large number of single fibre action potentials. The CAP recorded is the result of a large number of different fibres depolarising. The propagation velocity of the action potential on each fibre is determined largely by the diameter of that fibre. The CAP generated from the firing of a group of similar fibres is measured as a positive peak potential P1, then a negative peak N1, followed by a second positive peak P2. This is caused by the region of activation passing the recording electrode as the action potentials propagate along the individual fibres. An observed electrically evoked CAP signal from A3 fibres will typically have a maximum amplitude in the range of microvolts and a duration of 2-3 ms.

The CAP profile takes a typical form and can be characterised by any suitable parameter(s) of which some are indicated in FIG. 4. Depending on the polarity of recording, a normal recorded profile may take an inverse form to that shown in FIG. 4, i.e. having two negative peaks N1 and N2, and one positive peak P1.

In almost all neuromodulation applications, a single class of fibre response is desired, but the stimuli can recruit action potentials on other classes of fibres which cause unwanted side effects. Moreover, the difficulty of recording evoked neural responses has led to conventional solutions using proxy indicators such as observations of muscle contractions, without any knowledge of actual fibre type recruitment.

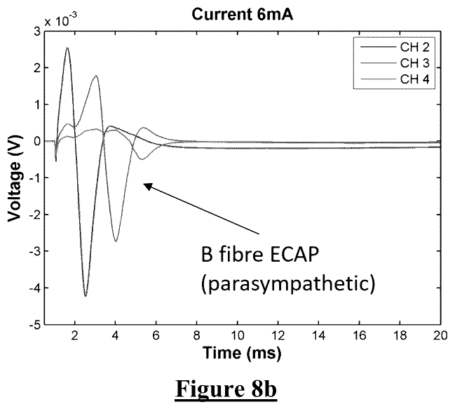

In accordance with the present embodiment, the CAP evoked by a given stimulus can be characterised by the parameters of the inflexion points in the curves of FIG. 4, FIG. 8a or FIG. 8b, for example. The positions and amplitudes of the peaks can be used alone or in combination to generate a correlation between them and the state and severity of a CNS disorder. Other electrophysiological data can be used to supplement the ECAP data. For instance, masker-probe studies can be used to determine the refractory period and the relative refractory period. The measurement of refractory periods allows an estimate of the frequency response of the fibres being stimulated. In particular, a shorter refractory period correlates with higher conduction velocity, thus allowing a determination of which fibre type(s) was/were recruited to give rise to the observed ECAP, and can thus provide a guide for setting stimulation frequency parameters. All these neurophysiological properties can be used to identify the stimulated nerves and can be used to guide fibre type targeting and stimulus parameter selection.

Almost all major nerves in the periphery are of mixed nature, meaning that the nerve contains fibres of various types and functions that run together. The peripheral nerves bundle together at various stages and form the spinal nerves (such as the S3 nerve, which is the main target for SNS). Before joining the spinal cord, the spinal nerves split up into the ventral and dorsal roots. In simplified terms, the ventral roots contain mostly a variety of efferent fibres, and the dorsal roots contain mostly a variety of afferent fibres. Mixed nerves therefore can contain both afferent and efferent axons. Another example of a mixed nerve is the vagus nerve (VN) which contains motor fibres, sympathetic fibres, parasympathetic fibres, and sensory fibres.

Mixed nerves are heterogenous collections of fascicles, and it has been shown that the fascicles bundle nerve fibres that serve similar functions and share common physiological properties. This separation of function can be observed from the rootlets which form the dorsal and ventral roots of each spinal nerve. FIG. 5 is a schematic representation of the functional separation of fibres in the ventral rootlets of a human S2 nerve in cross-section. The root consists of 2 rootlets. Three different nerve distribution patterns arise: the somatic type (S) with predominant large, thickly myelinated fibres and absence of parasympathetic fibres; the vegetative type (V) with abundance of parasympathetic fibres; and the mixed type (M). Note the topographic aggregation of the fascicles of vegetative and somatic types. The fascicles with predominance of parasympathetic fibres are concentrated in the right rootlet of either root. In contrast, purely somatic fascicles are found in the left rootlets. It appears that the nerve fibres do not simply follow a random distribution, but rather some sort of functional organization.

Stimulation of any given subsection of a mixed nerve, for example by applying stimuli only from one side of the nerve at an amplitude which only recruits fibres in fascicles proximal to the stimulus electrode, will therefore activate a particular portion of fibres that serve a distinct function. The present invention recognises that targeting the appropriate fibres of a mixed nerve is of great importance for many neuromodulation applications.

However, such targeting is in essence impossible from a purely anatomical approach, because the course of each fascicle in a mixed nerve varies along the nerve. The fascicles can cross, merge, and split. An example of this from the median nerve is given in FIG. 6, which is a three-dimensional (3D) reconstruction of median nerve fascicles. FIG. 6a is a 3D model of a median nerve at an arbitrary position, showing some changing patterns of different functional fascicles. Green represents motor nerve fascicles, yellow represents sensory nerve fascicles, and purple represents mixed (sensory and motor) nerve fascicles. FIG. 6b is an enlarged partial view of FIG. 6a, while FIG. 6c presents one transverse cross section image.

FIG. 6 demonstrates that fibre type targeting is difficult to address based only on anatomy because a desired fibre type takes significantly varying positions within the nerve at different parts of the nerve. Further, inter-patient variability of the content and disposition of fascicles within a nerve exists. These changes occur on scales which are significantly smaller than a typical neurostimulation electrode spacing, as typical implanted electrode arrays utilise electrodes which are 3 mm long, and are 4 mm apart from each other, i.e. positioned on a 7 mm pitch. However, in the space of 7 mm along the nerve shown in FIG. 6, any given nerve fascicle could take any or all positions within the bundle making it impossible to selectively target that fascicle based on surrounding anatomical orientations. Simplistically utilising smaller electrodes would not resolve these uncertainties.

Nevertheless, the present invention recognises that nerve fibres can be classified based on their physiological properties, such as myelination state and diameter. These properties result in differences in electrophysiological properties that allow the classification of stimulated nerve fibres through measures of, among others, the conduction velocity of the generated action potentials, their refractory period, and their strength-duration curves. For example, a linear relationship exists between the diameter of a myelinated fibre and the conduction velocity. A range of nerve fibre type classification systems exist, however regardless of nomenclature or classification used throughout this document, it is to be understood that it is the measures of differences in electrophysiological properties which permit differentiation of fibre types in accordance with the present invention, irrespective of nomenclature.

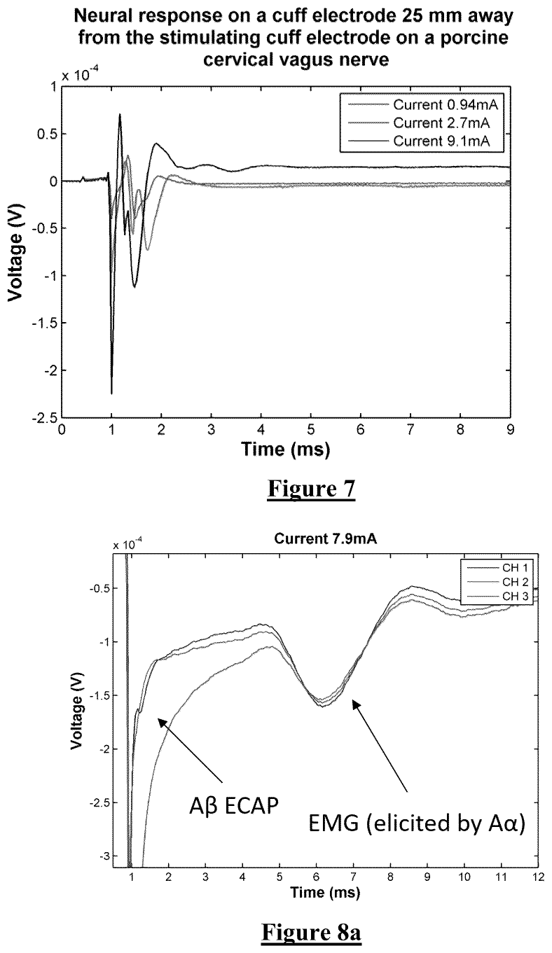

FIG. 7 shows the neural responses recorded from the cervical vagus nerve of a pig from a recording cuff electrode which was located about 25 mm away from the stimulating cuff electrode around the same nerve, for various currents.

FIGS. 8a and 8b show electrophysiological responses obtained from the S3 sacral nerves of 2 human patients undergoing SNS therapy. Although the electrodes were placed using the same surgical technique for each human patient, the observed responses were markedly different between FIG. 8a and FIG. 8b. The sacral responses of FIGS. 8a and 8b were obtained by stimulating from standard cylindrical electrodes therefore preferentially activating the fibres on the side of the nerve which was in closest proximity with the electrode. Note that in FIGS. 8a and 8b the three channels recorded denoted CHn, were obtained from consecutive electrodes along the lead with increasing distance from the stimulus electrode on the same lead (6 mm, 12 mm and 18 mm from stimulus, respectively), so that neural responses propagating as action potentials along a nerve occur later in time on the channels from more distal recording electrodes, whereas non-propagating signals are not spaced apart in time across the respective channels, allowing the present invention to distinguish neural responses from other electrophysiological activity. Note also that in FIG. 8a electrode 4 was used for stimulation, so that CH3 was closest to the stimulus site and CH1 was furthest from the stimulus site, in contrast to FIG. 8b in which electrode 1 was used for stimulation so that CH2 was closest to the stimulus site and CH4 was furthest from the stimulus site. The response amplitude in FIG. 8b is noted to be tens of times larger than that of FIG. 8a, despite a slightly smaller stimulus. Such variation is common in practice and may well be attributed to differences in the position of each respective electrode array relative to the nerve. The large amplitude responses in FIG. 8b illustrate how much power saving can be achieved by applying the present invention in order to reduce the stimulus as much as possible to the minimum level where the desired therapeutic effect is achieved, which may also have a further benefit of avoiding inappropriately high recruitment levels which may be painful, injurious, or could cause unwanted side effects.

In contrast the vagal responses of FIG. 7 were obtained by stimulating from cuff electrodes surrounding the nerve, so that all fibre types were recruited based on their thresholds independently of their location around the circumference of the nerve. This difference in recruitment based on nerve fascicle position explains the difference in the responses obtained from the vagus and the sacral nerves as seen when comparing FIG. 7 on the one hand to FIGS. 8a and 8b on the other hand.

In particular, in FIG. 7, at low currents only larger fibre types are recruited and the observed ECAP exhibits few peaks. As the current is increased, smaller fibres are recruited and the resulting ECAP starts to display additional peaks, in addition to the expected increase in peak amplitude.

On the other hand in FIG. 8a we observe an Aβ ECAP in the timeframe around 1-2 ms, and a myoelectric response. The ECAP in the timeframe around 1-2 ms can be specifically identified as an Aβ ECAP because of the conduction velocity and the distance of each respective recording electrode from the stimulus site. Further, the signal component observed in the timeframe around 4-10 ms can be specifically associated with an Aα response (either from direct activation or via a reflex arc by means of for example Ia fibre activation). While the fast conduction velocity of the Aα response mean that this neural response itself is obscured in the very early part of the recording (e.g. in the period <1 ms), the existence of an Aα response can nevertheless be noted because of the existence of the non-propagating signal component observed in the timeframe around 4-10 ms, which is an electrical field resulting from muscle activation and which therefore must arise due to Aα activation as this is the role of Aα fibres. In FIG. 8b we observe B fibres (most likely preganglionic parasympathetic efferents) dominating the response, and again this observed component of the recording can be specifically identified as a B fibre response because of the observed conduction velocity of the response past the three spaced recording electrodes being used. Although therefore likely to be B fibres, the responses could also stem from Aδ fibres, and it is again to be noted that the nomenclature used is not taken to be limiting to the fibre type differentiation enabled by the present invention.

The sympathetic and parasympathetic systems serve complementary roles in the modulation of visceral function. As a rule of thumb, the parasympathetic fibres are responsible for “rest or digest” response whereas the sympathetic fibres are responsible for “fight or flight” response. In the case of micturition for example, parasympathetic nerves excite the bladder and relax the urethra, whereas the sympathetic fibres inhibit the bladder body and excite the bladder base and urethra. In the case of bladder innervation, the preganglionic sympathetic fibres exit the spinal cord in the ventral roots at the rostral lumbar segments, the preganglionic parasympathetic fibres exit the spinal cord through the ventral roots of the sacral nerves. Accordingly, in some embodiments the present invention recognises that selectively targeting these fibre types may be of utility in therapy for incontinence, in contrast to prior approaches of targeting remote muscle responses as a proxy for therapy. Such embodiments of the invention may thus provide techniques for measuring evoked compound action potentials (ECAPs) from the sacral nerve to improve SNS both by better targeting the appropriate nerves, and by applying closed-loop stimulation in chronic implants based on the ECAP observations.

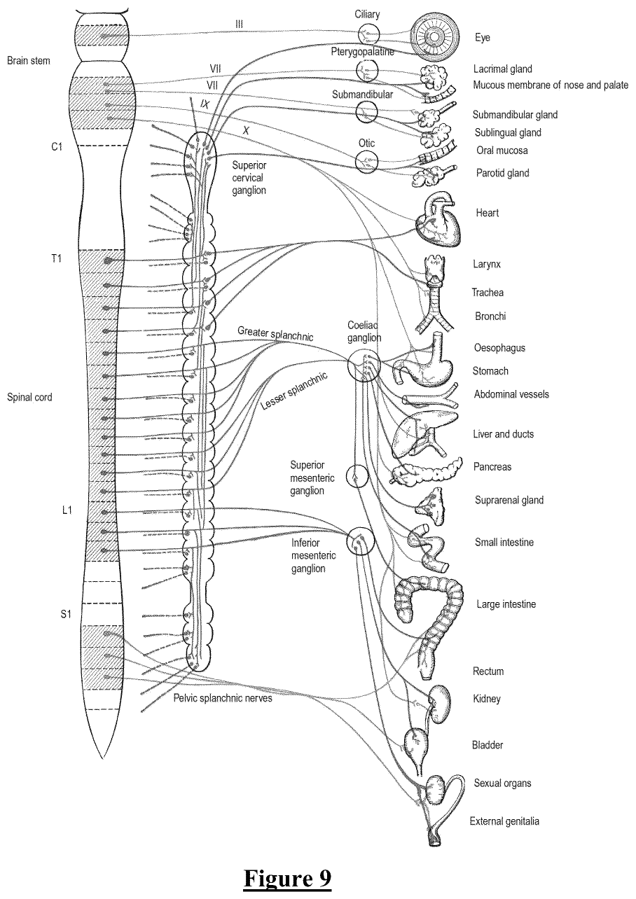

More generally, other embodiments of the invention may apply a similar targeted approach to neuromodulation upon any mixed nerve of the body in order to provide therapy for dysfunction associated with any such nerve. FIG. 9 illustrates a multiplicity of such sympathetic (red) and parasympathetic (blue) efferent pathways, which form part of mixed nerves upon which the approach of the present invention may be applied. The interrupted red lines indicate postganglionic rami to the cranial and spinal nerves.

Note that parasympathetic innervation of the heart, larynx, oesophagus, stomach, liver, pancreas, intestine, and kidney all originate from the 10th cranial nerve, also referred to as the vagus nerve. Parasympathetic innervation of the intestine, bladder, and genitalia originates from the sacral segments of the spinal cord. Note that the upper and lower parts of the large intestine are innervated from different parts, the upper part from the vagus nerve, the lower part from the sacral nerves.

The sacral nerve is a mixed nerve containing the full spectrum of fibre types and functions. It contains C fibres as well as myelinated fibres ranging from B (parasympathetic) to Aα motor neurons and carries both afferent as well as efferent neural signals. The sacral nerve is not homogenous and is made up of rootlets, further subdivided into fascicles, which preserve some degree of functional separation of the fibre types.

Nerve fibres are classified by their function as well as their physical properties (mostly myelination state and fibre diameter). It is important to note that this separation of physical properties and function is not absolute, and neural signals of a variety of functions are carried via fibres of similar properties (there are more functions than there are classes of fibres). As a rule of thumb, the diameter of myelinated fibres (in μm) is correlated to the conduction velocity (in m/s) by a factor of about 6. Thanks to this property, it is possible to use ECAPs measured from SNS electrodes to determine conduction velocity, and in turn to determine which fibres are being recruited by the stimulation paradigm. This can improve the therapy on 2 levels: aetiology-specific targeting of the appropriate nerves as well as closed-loop feedback control to maintain a stable therapy and improve effectiveness.

As the sacral nerves are non-homogeneous mixed nerves, the location of the lead with respect to the nerve fibres plays a large role in determining which fibre types are activated by SNS. Without intending to be being limited by theory, the recordings of the electrophysiological response made from SNS electrodes can differentiate whether muscle efferents, sensory afferents, somatic afferents and efferents, parasympathetic fibres, or C fibres are being activated. Both the ECAP properties as well as the late response properties can be used to determine which fibres are activated by the stimulation.

For example, in FIG. 8a the presence of a non-propagating late response indicates activation of Aα efferents (either directly or indirectly), and the presence of an Aβ response indicates sensory fibres are activated. In FIG. 8b, the presence of a B fibre response can indicate the activation of parasympathetic fibres. Due to the segmented nature of the sacral nerve (separation into fascicles in which bundles of fibres with similar function are clustered), the responses observed from SNS are not homogeneous (see FIG. 8). Further, inter-patient variability in the sacral plexus likely leads to variations in fibre types present in each sacral nerve.

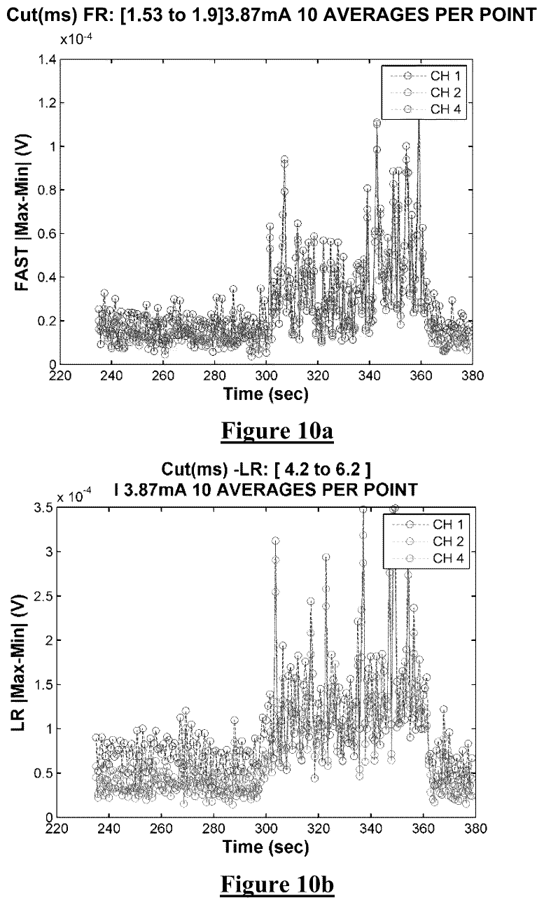

Independently of the type of response observed with SNS, changes in the patient's body posture as well as internal mechanisms that cause a movement of the electrode with respect to the nerve are reflected in a change in the amplitude of the neural response. FIG. 10a illustrates changes in amplitude of the Aβ response (example in FIG. 8a around 1-2 ms), denoted FAST in FIG. 10a. FIG. 10b illustrates changes in amplitude of the myoelectric response (example in FIG. 8a around 4-10 ms), denoted LR in FIG. 10b. This data was obtained for a patient undergoing a SNS trial in the S3 sacral nerve root. The patient was sitting at the beginning of the experiment of FIG. 10, stood up at around 300 seconds, and sat back down at around 360 seconds. The increase in amplitude of the neural responses, which approximately doubled from sitting to standing, was felt as an increase in paraesthesia sensation by the patient. A similar increase in amplitude was observed in the myoelectric response.

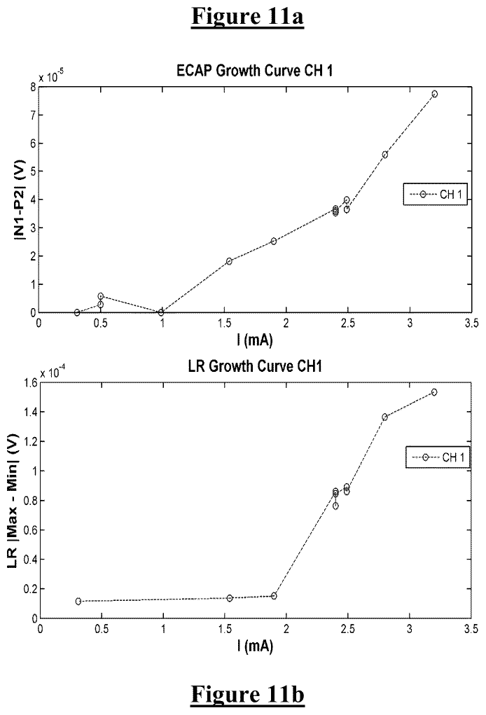

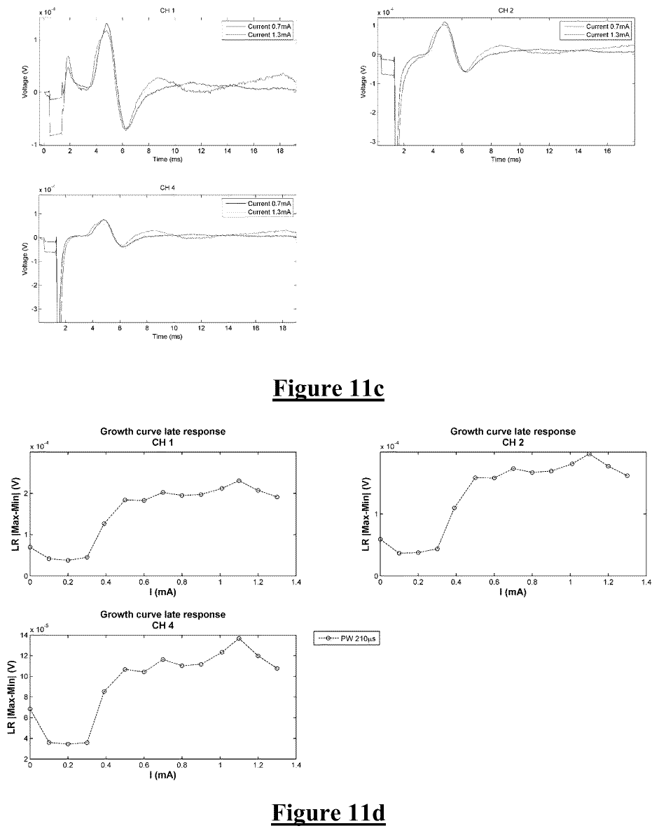

FIG. 11a illustrates measurements of the growth curve of the neural Aβ response of the same human SNS patient as FIG. 10. FIG. 11b illustrates measurements of the myoelectric response growth curve. The growth curve herein refers to measures of electrophysiological response amplitude in response to increasing stimulus intensity. Where a recording of an electrophysiological response comprises multiple components, such as comprising both an ECAP and a myoelectric response, separate growth curves of each such component may be obtained. Both the Aβ response of FIG. 11a and the myoelectric response of FIG. 11b display threshold behaviour, in that for the application of stimuli at current levels below a certain threshold, no response arises. The threshold in FIG. 11a by inspection appears around 1.5 mA, while the threshold in FIG. 11b by inspection appears at around 2 mA. Above the respective threshold each growth curve exhibits a linear section in which linearly increasing the stimulus current leads to an approximately linear increase in both the neural Aβ response and the myoelectric response. Both curves are expected to plateau at higher stimulus amplitudes as maximum recruitment is achieved, however these higher stimulus values were not applied in this experiment as it would have been painful to the patient. FIG. 11 thus illustrates that the myoelectric and Aβ responses each have a linear part which can be separately or jointly exploited by a feedback loop operating within the respective linear range shown in FIG. 11.

FIG. 11c illustrates recordings from a human patient treated for urinary incontinence with S3 sacral nerve stimulation. Clear late responses are visible on all recording channels CH1, CH2 and CH4, during the time period around 3-10 ms, at both 0.7 mA stimulation and 1.3 mA stimulation. A fast response (ECAP) is not visible in these recordings as the stimulus pulse width obscures the ECAP. FIG. 11d shows the growth curve for the late response amplitude in response to increasing stimulus amplitude, as measured at each respective recording electrode. As seen in FIG. 11d, this is an example of a plateauing myoelectric response. Such recordings enable a useful range of stimulation to be ascertained, as no therapeutic benefit occurs below the threshold, and no additional fibres are recruited beyond the plateau in the growth curve. FIG. 11e is a plot of a plateauing growth curve for a slow response thought to be a B fibre response. Indicated on the curve is the part of the growth curve at which the patient started to feel the stimulation. Notably, this patient threshold was at the plateau of this B fibre response, meaning that the slow B fibre response observed in FIG. 11e was not linked to sensation, and that a first fibre type was recruited without recruiting others.

Some embodiments may further provide for staged fibre targeting, whereby in a first stage of operation a level of recruitment of a desired fibre type is monitored and maintained within a desired range of the curve of FIG. 11a, 11b or 11e, and wherein a second stage of operation is adopted when recruitment or observation of the desired fibre type is lost (as can be common with postural changes or lead migration), in which a recruitment of a secondary fibre type is instead monitored and maintained within a desired range so as to ensure continued neural recruitment in general even when recruitment of the selected fibre type in particular becomes untraceable. An alternative response to a loss of recruitment or observation of the desired fibre type may be to trigger any other suitable event, such as a warning signal or an automatic reprogramming procedure. Additionally, linearity testing may be undertaken in response to a loss of recruitment or observation of the desired fibre type, whereby the stimulation amplitude is varied to explore whether the recruitment responds linearly; absence of a linear response of recruitment to such stimulus amplitude variations may indicate a loss of therapeutic efficacy which may be used to trigger reprogramming or a warning signal or the like.

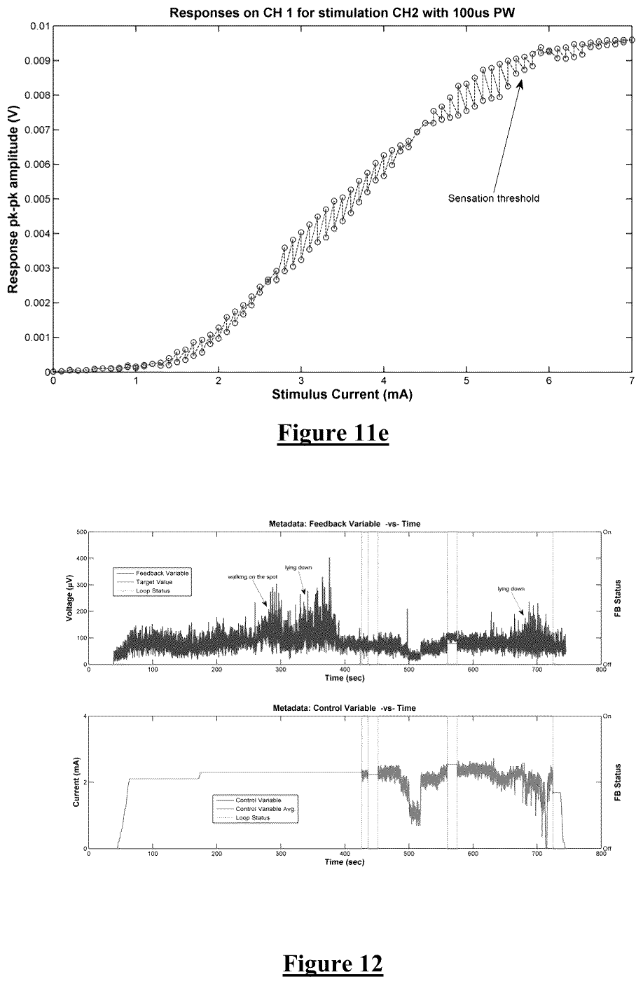

As noted in relation to FIG. 8b, it is also possible to identify recruitment of, and selectively target recruitment of, B fibres of the autonomic nervous system. The myoelectric response may similarly be targeted. FIG. 12 illustrates changes in the amplitude of myoelectric responses observed in a human SNS patient over time (upper trace), together with the associated control variable (lower trace) over a period of time covering both open and closed loop modes. FIG. 12 thus illustrates successful closed loop control based on a myoelectric response.

Equipped with these insights, embodiments of the invention may thus provide for aetiology specific fibre targeting in a mixed nerve. For example, in the case of SNS, instead of relying on past approaches utilising muscle responses without knowledge of fibre types recruited, targeting and programming can be performed using the invention. Aetiology specific fibre targeting in a mixed nerve recognises that in most cases when stimulating a mixed nerve, only a subset of fibres are relevant to the condition being treated by stimulation. Stimulating all fibres of the mixed nerve will inevitably lead to unwanted side effects or inefficient stimulation.

In the broadest sense, these particular embodiments optimise neuromodulation therapy on a mixed nerve by using electrophysiological measurements to selectively target a subset of fibre types in a mixed nerve. This will consist in optimising the stimulus parameters such that the responses of desired fibre types are obtained whilst minimising the responses of unwanted fibre types. In the ideal case, only the desired fibres will be activated by the device, although in many embodiments simply achieving preferential targeting of the desired fibre type(s) and/or preferentially minimising recruitment of other fibre types may nevertheless deliver the benefits of the present invention.

In one embodiment, this invention proposes a device that can stimulate and record electrophysiological signals obtained from electrodes placed near a mixed nerve. The neural response can then be analysed to indicate the nature of the stimulated fibres. In an example, the electrophysiological response may indicate the presence or absence of specific nerve fibres. Based on the desired therapeutic outcome, this information is used to optimise the therapy. For example, various parameters for administering the therapy such as, but not limited to, stimulation waveform and lead position may be adjusted to recruit the desired fibre types. In some cases, several causes can be treated simultaneously, each requiring stimulation of a different subset of fibres (such as B fibres for incontinence and sensory fibres for pelvic pain, both located in the sacral nerve). In this case, the method and device will use electrophysiological measurements to continuously optimise the delivered stimulation regime, in order to elicit responses of the desired fibres while minimising the responses from unwanted fibres.

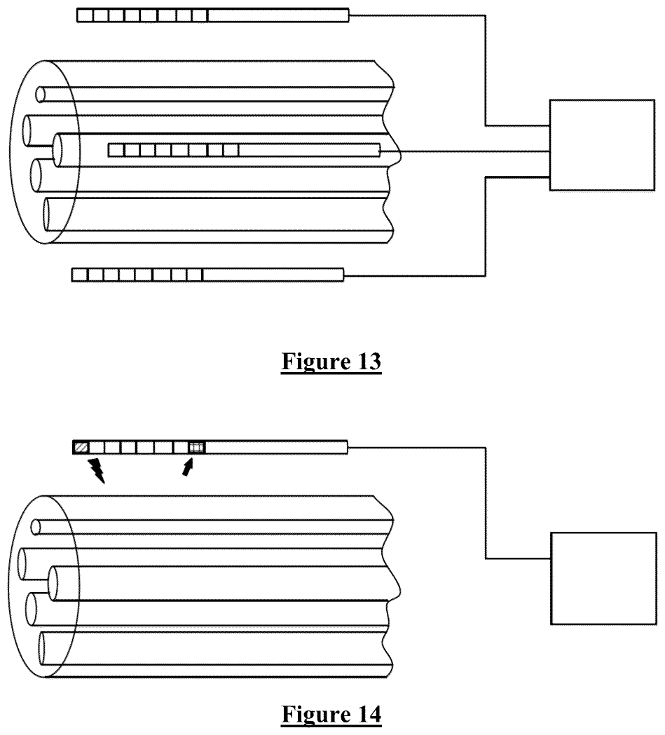

In one embodiment, shown in FIG. 14, we propose a method consisting of 2 or more electrodes implanted near a target mixed nerve of which only a subset of fibres are desired stimulus targets, and an implantable control unit device capable of recording the electrophysiological response to the stimulation. The stimulated fibre populations are assessed via the recorded response and the electrode placement, the electrode selection, as well as the stimulus parameters (including, but not limited to, pulse width, stimulus frequency, stimulus waveform, stimulus amplitude) are optimised such that the desired fibre types are preferentially targeted. Additionally, the recorded responses can be used to optimise stimulus parameters in order to minimise power consumption. As long as the desired responses are observed, the parameters requiring the least power to achieve this should be used. In one SNS trial, B fibres were observed to be fully recruited at a stimulus amplitude which was only around a quarter of the amplitude being used as defined by reference to the past approach of the muscle activation threshold. This observation indicates that therapeutic benefits provided by B fibre recruitment could be achieved at a fraction of the power of current state of the art SNS techniques which operate blind to fibre type.

The characteristics of the neural response (evoked compound action potential, or ECAP) are used in order to assess which fibre types are activated by the stimulus paradigm. Without being limited by theory, the ECAP characteristics that can be used to assess the type of the stimulated fibres includes the conduction velocity (including latency), strength-duration characteristics, and the refractory period. Additionally, myoelectric responses (Late Responses (LRs)) can be used as a proxy measure for Aα efferent activation (either directly or indirectly) based on their presence and latency.

For example, in the case of sacral nerve stimulation, targeting parasympathetic fibres (lightly myelinated B type fibres), the electrodes are placed near the S3 sacral nerve as shown in FIG. 14. A first electrode is used for stimulation and neural recordings are obtained from 1 or more electrodes of the same array, close to the target nerve. The conduction velocity of the stimulated fibres is obtained from the neural recordings and the electrodes are placed such that the B fibres response is maximised and all other responses minimised. Further, the stimulation parameters are varied such that the B fibre responses are maximised and all other responses minimised.

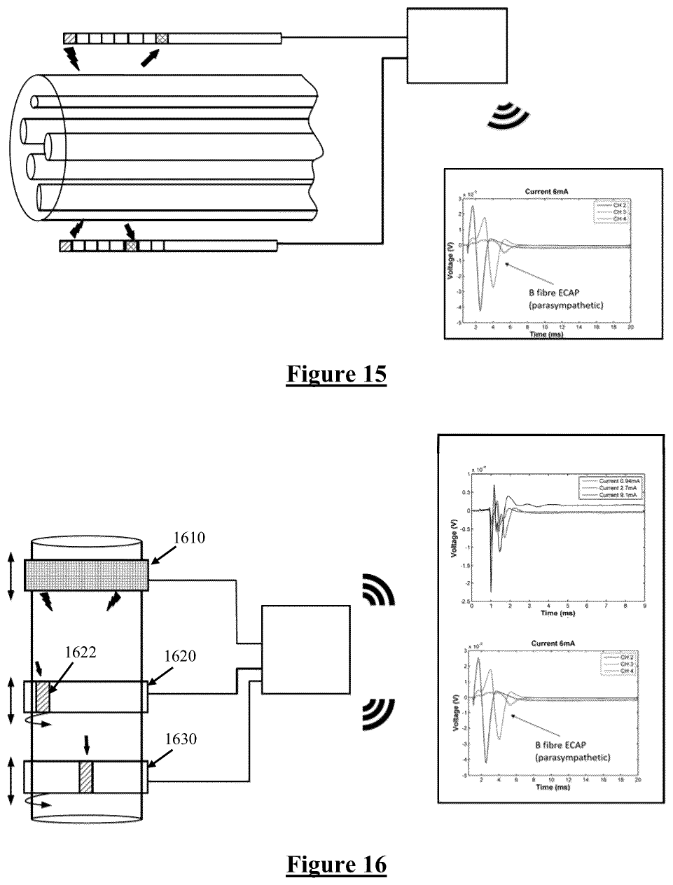

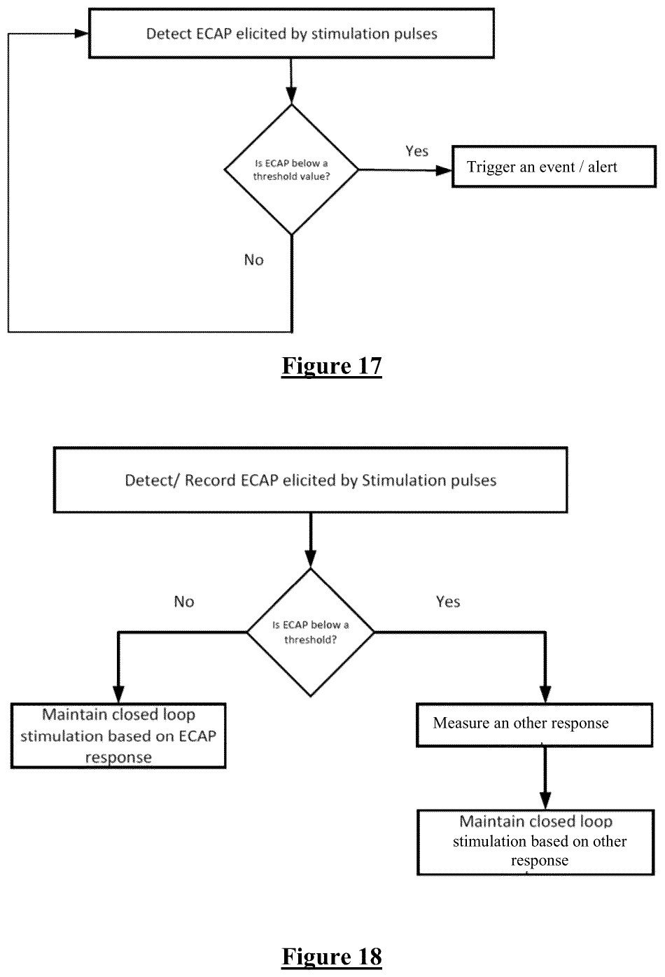

In other embodiments, such as those of FIGS. 13 and 15, a device is provided to optimise lead positioning and stimulus parameter selection for stimulation of a mixed nerve using a plurality of electrodes with different characteristics. This uses more than 1 lead placed near the target nerve. The selection of stimulus parameters is then carried out for more than 1 lead. In the case of a multitude of conditions, such as overactive bladder (OAB) as well as pelvic pain, electrode selection will be done so that either 1 or several electrodes on either one or several leads maximises the B fibre activation (for OAB relief) and the Aβ fibre activation (for pain relief). Once again, it is to be noted that the present invention is not limited by theory of mechanism as the fibre type targeting may be performed in respect of any suitable fibre which achieves a therapeutic benefit irrespective of the theorised mechanism of action.

In other embodiments, the nerves of a plexus can be stimulated in turn to determine which branch of the plexus contains the desired fibres using recordings of evoked electrophysiological responses. The therapy is then optimised on the best candidate branch of the plexus.

Furthermore, the recording of ECAPs for a given stimulus electrode does not have to be done on the same lead. The recording site can be optimised (maximising signal amplitude) and any electrode close to the target nerve can be chosen. This is useful as the fascicles in mixed nerves do not run parallel inside the nerve (as is shown simply for illustrative simplicity in FIGS. 13 to 15, but cross, merge, and split as shown in FIG. 6.

Another embodiment of this device will consist in implanting cuff electrodes instead of epidural leads, as shown in FIG. 16. The cuff electrodes will consist of several distinct contacts each (as opposed to some cuff electrodes that have one continuous electrode running along the entire cuff), and the 2 or more cuff electrodes can be rotated and moved up and down along the mixed nerve to find a position that optimises the discrete stimulation and recording of separate fascicles. In one embodiment the arrangement of FIG. 16 can be used for spatial targeting of a selected fibre type. This involves the cuff electrode 1610 applying a supramaximal stimulus around substantially an entire circumference of the nerve, in order to recruit all fibres of the nerve. Electrode 1610 may be a non-segmented electrode extending continuously around the nerve or may be a segmented electrode with the stimulus being applied simultaneously by all segments of the electrode 1610 to ensure all fibres are recruited. The recruited responses resulting from the stimulus can then be observed at selected circumferential positions by using a selected segment (e.g. segment 1622 of a segmented cuff electrode 1620 (other segments not shown for clarity). The response observed at segment 1622 in particular can then be analysed to determine a dominant fibre type which is adjacent to that segment 1622. This knowledge can then be used to identify which fibre type(s) are adjacent all segments of each segmented cuff electrode. In turn, this knowledge can be used to preferentially use a given segment, such as segment 1622, to deliver stimulation which will preferentially recruit the fibre type which is known to be adjacent to that segment. Other embodiments may derive the same knowledge by instead using segment 1622 to apply stimuli which are only just above a stimulus threshold and which therefore only recruit fibres proximal to segment 1622. The responses may then be observed at cuff electrode 1610 and analysed to determine the type of fibres being recruited by segment 1622 and thus the types of fibres which are adjacent to 1622. A survey may be carried out over multiple segments to thereby determine the fibre type(s) adjacent to all such electrode segments, and spatial fibre type targeting may then be applied by using such knowledge as a lookup table.

Additionally, the responses can be used to optimise stimulus parameters in order to minimise power consumption. As long as the desired responses are observed, the parameters requiring the least power to achieve this should be used.

More generically, different lead shapes can be used, paddle leads, percutaneous leads, cuff electrodes, while effecting fibre type targeting in accordance with the present invention.

In some embodiments, the device is connected externally to the electrodes, in other embodiments, the device is fully implantable. In some embodiments, the electrodes can be on a single lead, or multiple leads placed in proximity to the stimulus target, each lead can contain a multitude of electrodes. In other embodiments, some electrodes can be located distally to the target nerve to serve as reference electrode for the measurement or return electrode for the stimulation. In the case where the device is a fully implantable system, the body of the implant can be used as one electrode.

In some embodiments, the leads can be epidural, similar to those used in sacral nerve stimulation or spinal cord stimulation. In other embodiments, the leads can be paddle leads, or cuff electrodes. In some embodiments, the electrodes can further be segmented. In one example, a cuff electrode can in some cases be a ring electrode surrounding the nerve, and in some cases it can be a segmented ring whereby electrically distinct portions of the cuff electrode take distinct positions around the circumference of the nerve, allowing distinct circumferential positions around the nerve to be selectively targeted by stimulating from a selected segment or to be selectively observed by recording from a selected segment. In one example, an epidural lead can have circular electrode contacts, in other examples the contacts can be segmented.

In some embodiments, the stimulation and recording are performed on the same electrodes, in other embodiments the stimulation and recording are done on distinct electrodes whether on the same electrode lead or electrode array, or a different electrode lead or array. In some embodiments, some electrodes implanted near the target span an area small enough to avoid merging or branching of fibre tracts within the nerve. For example, in some embodiments, electrodes implanted near the sacral nerve can span a distance of less than 5 centimetres, preferably less than 2 centimetres.

In some embodiments, the external device may be used to analyse neural response during lead insertion. The external device may be used to deliver stimulation pulses to a target nerve in a patient. The external device may include a control unit, a display module and a stimulation module. The stimulation unit is configured to deliver stimulation pulses to a desired nerve and analyse the neural response generated. Thereafter, the control unit is adapted to analyse the neural response and differentiate the recruited nerves based on one or more parameters. The control unit is configured to transmit information to display the neural recruitment in one or more modes. In a first mode, the neural recruitment is displayed in the form of a collection of ECAP waveforms and myoelectric waveforms. In a second mode, the neural recruitment is displayed as waveforms which are labelled with the corresponding fibre types. In a third mode, the neural recruitment is displayed as waveforms with timing partitions to indicate the fibre types which were recruited. In a fourth mode, the neural recruitment is displayed along with characteristics of the fibres such as conduction velocity etc.

In some embodiments, the external device is adapted to operate one or more electrodes. The external device is adapted to deliver pulses via paddle leads, cylindrical leads and cuff electrodes. Further, the external device is configured to operate in any combination of the aforementioned electrodes.

Implantable device: In some embodiments, the implantable device is configured to perform all the functions as the external device except that it is implanted within the body of the patient.

In some embodiments, the electrophysiological responses elicited by the stimulation can be displayed in real time. In some embodiments, these responses can further be processed to increase the signal to noise ratio. For example, averaging can be used to decrease the random noise found on each individual response, and fit-and-remove algorithms can be used to decrease the artefact component in the signal. These signals can be used to recognise the activation of a certain fibre type or certain fibre types. The stimulus parameters can then be modified whether by trial and error, brute force exploration or any suitable search technique of the stimulus parameter space, so as to optimise fibre activation of the desired subsets of fibres as indicated by the displayed responses.

In some embodiments, the axes of the electrophysiological response graph can be labelled to facilitate the interpretation of the responses. For example, based on the stimulus and recording electrode configurations, the axes of the response graph can be labelled to indicate the times at which the response of a given fibre type should be recorded if present.

In some embodiments, the neural responses can be marked and/or labelled to help identify the fibre types being recruited by the stimulation. For example, the peaks of the response can be labelled with their time and amplitude parameters.

In some embodiments, a change in morphology of the response with varying stimulus parameters can be used to identify fibre type contributions in an ECAP in which the peaks are poorly defined. In one example, the current is slowly ramped up and the ECAP shape observed or automatically processed. Initially, the largest fibres will respond to external stimuli first, a change in ECAP morphology at higher amplitudes could indicate the recruitment of a second, slower fibre type. In another example, the frequency of the stimulation can be increased to an appropriate level, and the responses observed over time. The small fibres will fatigue first, the larger fibres last. The change in morphology over time can be used to determine which part of the ECAP is associated with which fibre type. Further, a decrease in amplitude of the myoelectric response at increasing stimulus frequency could be due to muscle fatigue, or depression of the H-reflex. These properties and others can be used to determine the fibre types being stimulated.

In some embodiments, processing can be done on the recorded neural responses to display an estimate of the conduction velocity of the different elements of the recording of the electrophysiological response.

In some embodiments, the recording(s) of the electrophysiological response can be processed such that an estimate of the number of fibres of each type being recruited is displayed. The estimate could in some instances give a proportion of fibre types or an absolute estimate of the number of fibres being activated.

In some embodiments, stimulation can be performed in a way to approach a constant neural recruitment by adapting the stimulus parameters automatically in a feedback arrangement in order to approach a target electrophysiological response. In one example, B fibres are preferentially targeted in a mixed nerve. After selecting the stimulus electrodes and parameters that optimise B fibre recruitment, the current (or another parameter) could be automatically adjusted to maintain a constant response amplitude. In another example, the late response can be used as a proxy measure for motor fibre activation, the stimulus can then be automatically adjusted to maintain a constant late response amplitude.

The amplitude of the electrophysiological response or a part of it could be done by assessing peak amplitudes, peak-peak amplitudes, the area under the curve, a convolution with a filter, or any other method.

In some embodiments, an implantable device may be configured to establish closed-loop stimulation on a targeted fibre type. For instance, the feedback loop may be established on the neural response of the B fibres in a mixed nerve. The methods disclosed herein enable the implantable device to establish a feedback loop on the neural response of certain fibre types. Since the implantable device is configured to determine the fibre types based on the neural response, the implantable device can maintain closed loop stimulation based on the response of the targeted fibre type. The implantable device may maintain closed-loop stimulation on at least one or more of the fibre types which include, but not limited to, Aα, Aβ, Aδ, B and C fibres. In some cases, closed-loop stimulation may be maintained on the myoelectric response elicited by the stimulation. If the desired response is not observable, another response can be used as a proxy measure for establishing and maintaining closed-loop stimulation.

In some embodiments, the neural recruitment can be automatically monitored for large variations that could be indicative of, for example, lead migration. In one example, a change in electrode position that would lead to loss of efficacy could be tested by the device by varying the current amplitude to a small degree and monitoring the response profile. If no change is observed with variations in stimulus amplitude, a warning signal is sent to the user, or stimulation is stopped or other suitable action occurs.

In some further embodiments, the implantable device may be configured to check the fibre types which are recruited by the stimulation pulses. This feature may be used to ensure that the desired fibre types are recruited for the majority of the stimulus duration. In some cases, when the implantable device fails to detect the response of a fibre type for a duration which exceeds a threshold, the implantable device may trigger an alert to the patient that the desired fibre type is no longer recruited. In such a case, the physician may consider the possible causes and take remedial action. In other embodiments, alternative actions may be triggered in such an event, such as automatic reconfiguration of the stimulus paradigm. Such functions may alternatively be carried out by an external device such as a clinician's programmer device.

In some embodiments, continuous monitoring can be used to both maintain a near constant neural recruitment, as well as to warn of a substantial change in lead placement or nerve properties. Further embodiments may provide for testing the feedback loop integrity before applying the next corrective stim pulse. This could be done by making sure that if the current is increased, the response also increases. This would keep the loop from diverging when the signal is lost (as can occur when the signal is lost when changing posture, or when the charger or another interfering source is applied).

In some embodiments, the method and device may be applied to vagus nerve stimulation, dorsal root stimulation, sacral nerve stimulation, or ventral root stimulation. In some embodiments, the method and device can be used in more than one location, simultaneously or in tandem. For example, the device could be used in the sacral nerve and the lower thoracic or upper lumbar ventral roots which innervate the bladder and bowel. Without being limited by theory, it is proposed that such a device can stimulate both the sympathetic and parasympathetic nervous system in locations specific for bladder and bowel control. Stimulating the ventral roots of the spinal cord and/or the vagus nerve, and/or the sacral nerve can lead to similar embodiments in which the target is different from the bladder and bowel, such as any one or more of the heart, larynx, trachea, bronchi, oesophagus, stomach, liver, pancreas, small intestine, spleen, large intestine, kidney or sexual organs. For example, sympathetic and parasympathetic stimulation of the liver could be achieved by stimulating the sympathetic fibres in the thoracic sections T5-T12 and the parasympathetic fibres originating in the vagus nerve. Vagus nerve stimulation may be improved by some embodiments of the present invention may selectively avoiding stimulation of fibre types which in past solutions cause fatigue of the throat, swallowing muscles, vocal chords, and the like, which in past solutions are unnecessarily recruited. Note also partial lesions of the spinal cord might be best healed by targeting specific fibres.

In other embodiments the programming and display module does not have to be part of the implanted device. In such embodiments the implanted stimulator itself only executes the program that was set by the control module, and the responses are observed in real time or later only on an external programmer, and not on the implanted device.

The preceding thus reveals large inter-patient variability in fibre type recruitment and significant posture-related effects on the number of fibres recruited. Further, there was a clear distinction between fibre type recruited and trial outcome. These first-in-human results show that recordings of an electrophysiological response can be used to identify the type of fibres recruited by SNS therapy and that the type of fibre may correlate with therapeutic effectiveness. These results pave the way for improved targeting as well as closed-loop SNS that has the potential to greatly improve the therapy.

Notably, the fibre targeting of the present invention allowed some subjects to achieve effective therapy at stimulation levels which were orders of magnitude less than a muscle response threshold or sensory response threshold which would conventionally have been used to set a stimulation level. This would lead to a many multiples increase in battery lifetime.

Embodiments undertaking fibre type targeting on an ongoing basis to control a feedback process have the further advantage of being responsive to lead movement relative to the nerve, as occurs with posture changes, coughs, sneezes and the like. In contrast to conventional approaches which fix the stimulation at a single high level, the use of feedback based on electrophysiological measurements allows stimulation to be constantly revised to ensure that the desired fibre type continues to be recruited by suitably refining the stimulation parameters on an ongoing basis. Similar benefits can be obtained even in simpler embodiments which simply detect a loss of recruitment of the desired fibre type and issue an alert to the user to, for example, seek clinical assistance. Alternatively, automatic modification of the stimulus paradigm could be performed in some embodiments in the event in which the determined stimulus paradigm fails to recruit the desired fibre types.

It is to be understood that the fibres types named in the examples are based on current understanding and should not be considered limiting in the application of the present invention.

The described electronic functionality can be implemented by discrete components mounted on a printed circuit board, and/or by a combination of integrated circuits, and/or by an application-specific integrated circuit (ASIC).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

1. A method of neurostimulation of a mixed nerve comprising a plurality of nerve fibre types, the method comprising:
delivering to the mixed nerve an electrical stimulus from at least one implantable nominal stimulus electrode, the electrical stimulus being delivered in accordance with a set of stimulus parameters;
obtaining from at least one implantable nominal recording electrode a recording of an electrophysiological response evoked by the electrical stimulus;
analysing one or more selected characteristics of the recording to determine whether a recruitment of a first fibre type by the electrical stimulus has occurred; and
based on the recruitment determination, refining the stimulus parameters in a manner to effect selective recruitment of the first fibre type relative to other fibre types of the mixed nerve.

2. The method of claim 1 wherein the one or more selected characteristics comprise propagation or non-propagation of the recording of the electrophysiological response.

3. The method of claim 1 wherein the stimulus parameters are refined to effect selective recruitment of the first fibre type while further effecting selective non-recruitment or diminished recruitment of at least one other fibre type.

4. The method of claim 1 wherein the stimulus parameters are varied and a change in morphology of the electrophysiological response with the varied stimulus parameters enables determination of whether recruitment of the first fibre type or the other fibre types has occurred.

5. The method of claim 4 wherein the electrophysiological response comprises a neural response without defined peaks.

6. The method of claim 4 wherein the varied stimulus parameters comprise a stimulation current and the one or more selected characteristics comprise evoked compound action potential (ECAP) shape, and the method further comprises incrementally increasing the stimulation current and observing a change in the ECAP shape resulting from the incrementally increasing stimulation current.

7. The method of claim 6 wherein observing the change in ECAP morphology at higher amplitudes enables determination that a second fibre type is recruited.

8. The method of claim 4 wherein the varied stimulus parameters comprise stimulation frequency, and the one or more selected characteristics comprise an evoked compound action potential (ECAP) shape, and the method further comprises increasing the stimulation frequency and observing a change in the ECAP shape.

9. The method of claim 1 wherein the method comprises assessing a plurality of the selected characteristics of the recording to determine whether the recruitment of the first fibre type by the electrical stimulus has occurred, the plurality of the selected characteristics of the recording comprising two or more of: one or more electrophysiological response inflexion points; one or more electrophysiological response peak positions; one or more electrophysiological response peak amplitudes; electrophysiological response propagation velocity; propagation or non-propagation of the electrophysiological response; electrophysiological response duration; refractory period; strength-duration curve characteristics including chronaxie or rheobase; growth curve characteristics including threshold and slope; number of electrophysiological response peaks with increasing stimulus current; presence, amplitude or latency of a late response; response properties of the electrophysiological response to varying stimulus frequencies; zero crossings of the electrophysiological response; a width of lobes of the electrophysiological response.

10. The method of claim 1 wherein the recording of the evoked electrophysiological response comprises a myoelectric response in addition to a neural response.

11. The method of claim 10 wherein the method comprises assessing the one or more selected characteristics of each of the myoelectric response and the neural response to determine whether the recruitment of the first fibre type by the electrical stimulus has occurred.

12. A neurostimulation device comprising:
a control unit configurable to:
deliver to a mixed nerve an electrical stimulus from at least one nominal stimulus electrode of a plurality of electrodes, the electrical stimulus being deliverable in accordance with a set of stimulus parameters;
obtain from at least one nominal recording electrode of the plurality of electrodes a recording of an electrophysiological response evoked by the electrical stimulus;
analyse one or more selected characteristics of the recording to determine whether a recruitment of a first fibre type by the electrical stimulus has occurred; and
refine the stimulus parameters, based on the recruitment determination, in a manner to effect selective recruitment of the first fibre type relative to other fibre types of the mixed nerve.

13. The device of claim 12, wherein the one or more selected characteristics comprise propagation or non-propagation of the recording of the electrophysiological response.

14. The device of claim 12 wherein the control unit is further configurable to refine the stimulation parameters to effect selective recruitment of the first fibre type while further effecting selective non-recruitment or diminished recruitment of at least one other fibre type.

15. The device of claim 12 wherein the control unit is further configurable to vary the stimulus parameters, and a change in morphology of the electrophysiological response with the varied stimulus parameters enables determination of whether recruitment of the first fibre type or the other fibre types has occurred.

16. The device of claim 15 wherein the varied stimulus parameters comprise a stimulation current and the one or more selected characteristics comprise evoked compound action potential (ECAP) shape, and the control unit is further configurable to incrementally increase the stimulation current and observe a change in the ECAP shape resulting from the incrementally increasing stimulation current.

17. The device of claim 16 wherein observing the change in ECAP morphology at higher amplitudes enables determination by the control unit that a second fibre type is recruited.

18. The device of claim 15 wherein the varied stimulus parameters comprise stimulation frequency, and the one or more selected characteristics comprise evoked compound action potential (ECAP) shape.

19. The device of claim 12 wherein the control unit is configurable to assess a plurality of the selected characteristics of the recording to determine whether the recruitment of the first fibre type by the electrical stimulus has occurred, the plurality of the selected characteristics of the recording comprising two or more of: one or more electrophysiological response inflexion points; one or more electrophysiological response peak positions; one or more electrophysiological response peak amplitudes; electrophysiological response propagation velocity; propagation or non-propagation of the electrophysiological response; electrophysiological response duration; refractory period; strength-duration curve characteristics including chronaxie or rheobase; growth curve characteristics including threshold and slope; number of electrophysiological response peaks with increasing stimulus current; presence, amplitude or latency of a late response; response properties of the electrophysiological response to varying stimulus frequencies; zero crossings of the electrophysiological response; and a width of lobes of the electrophysiological response.

20. The device of claim 12 wherein the recording of the evoked electrophysiological response comprises a myoelectric response in addition to a neural response, and the control unit is configurable to assess the one or more selected characteristics of each of the myoelectric response and the neural response to determine whether the recruitment of the first fibre type by the electrical stimulus has occurred.

21. A method of neurostimulation of a mixed nerve comprising a plurality of nerve fibre types, the method comprising:
delivering to the mixed nerve an electrical stimulus from at least one implantable nominal stimulus electrode, the electrical stimulus being delivered in accordance with a set of stimulus parameters;
obtaining from at least one implantable nominal recording electrode a recording of an electrophysiological response evoked by the electrical stimulus;
analysing one or more selected characteristics of the recording to determine whether a recruitment of a first fibre type by the electrical stimulus has occurred; and
based on the recruitment determination, issuing an alert.

22. A neurostimulation device comprising:
a control unit configurable to:
deliver to a mixed nerve an electrical stimulus from at least one nominal stimulus electrode of a plurality of electrodes, the electrical stimulus being deliverable in accordance with a set of stimulus parameters;
obtain from at least one nominal recording electrode of the plurality of electrodes a recording of an electrophysiological response evoked by the electrical stimulus;
analyse one or more selected characteristics of the recording to determine whether a recruitment of a first fibre type by the electrical stimulus has occurred; and
issue an alert based on the recruitment determination.
